(19) United States
(12) Patent Application Publication
(10) Pub. No.: US 2023/0125734 A1
(43) Pub. Date: Apr. 27, 2023

(54) MEDICAL INSURANCE METHOD

(71) Applicant: SONY GROUP CORPORATION, TOKYO (JP)

(72) Inventors: TAKAYOSHI HIRAI, TOKYO (JP); YOHEI EBIHARA, TOKYO (JP); NAOTO NAKAMURA, TOKYO (JP); HIRONORI SUZUKI, TOKYO (JP)

(21) Appl. No.: 17/906,269

(22) PCT Filed: Mar. 12, 2021
(86) PCT No.: PCT/JP2021/010243

§ 371 Date: Sep. 14, 2022

(30) Foreign Application Priority Data
Mar. 24, 2020 (JP) 2020-053178

(51) Int. Cl. G06Q 40/08; G16H 10/60

(52) U.S. Cl. CPC G06Q 40/08; G16H 10/60

(57) ABSTRACT

A medical insurance method according to the present disclosure includes a step of acquiring, as a first patient condition, a type of a condition of a patient estimated through input of first patient data of the patient to a predetermined machine learning model, a step of acquiring, as a second patient condition, a type of a condition of the patient estimated by a method different from the predetermined machine learning model on the basis of data including the first patient data, a step of acquiring a third patient condition estimated on the basis of second patient data measured after measurement time of the first patient data, and a step of determining whether or not insurance coverage is available on the basis of the first patient condition, the second patient condition, and the third patient condition.

### TECHNICAL FIELD

The present disclosure relates to a medical insurance method.

### BACKGROUND ART

In recent years, utilizing an artificial intelligence (AI) system including machine learning, as an auxiliary tool for doctors or a diagnostic tool instead of doctors has been proposed. For example, utilizing pathological diagnosis by AI as a second opinion for pathologists has been considered. Furthermore, it has been also proposed that patients utilize diagnosis by AI as preliminary diagnosis. For example, a home medical support system that calculates an insurance premium is provided (see Japanese Patent Application Laid-Open No. 2019-53789).

### CITATION LIST

### Patent Document

Patent Document 1: Japanese Patent Application Laid-Open No. 2019-53789

### SUMMARY OF THE INVENTION

### Problems to be Solved by the Invention

According to a conventional technique, an insurance premium is calculated on the basis of medical treatment provided in home, such as drip infusion or medication, and contents of an in-home management guidance, such as meal support or nursing care support.

However, not only diagnosis by human but also by AI can be wrong. In such cases, each county has different discussion on whether users (for examples, doctors or patients) or companies that developed the AI should be responsible for wrong diagnosis by AI. Under the circumstances, users may cease active use of AI due to fear of effects of wrong diagnosis by AI.

Furthermore, in a case where companies are responsible for wrong diagnosis by AI, motivation of companies that have been engaged on AI decreases. Thus, preparations for wrong diagnosis by AI are demanded to promote active use of AI in a medical field. In view of the circumstances as described above, systems and the like for promoting utilization of AI has been desired.

Therefore, the present disclosure proposes a medical insurance method that can promote use of AI in a medical field.

### Solutions to Problems

In order to solve the above issue, one aspect of a medical insurance method according to the present disclosure includes a step of acquiring, as a first patient condition, a type of a condition of a patient estimated through input of first patient data of the patient to a predetermined machine learning model, a step of acquiring, as a second patient condition, a type of a condition of the patient estimated by a method different from the predetermined machine learning model on the basis of data including the first patient data, a step of acquiring a third patient condition estimated on the basis of second patient data measured after measurement time of the first patient data, and a step of determining whether or not insurance coverage is available on the basis of the first patient condition, the second patient condition, and the third patient condition.

### BRIEF DESCRIPTION OF DRAWINGS

FIG. 1 is a schematic view of a pathological AI system.

FIG. 2 is a view illustrating an outline of an example insurance system.

FIG. 3 is a view illustrating an example table.

FIG. 4 is an example flowchart of the insurance system.

FIG. 5 is an example flowchart of a modification.

FIG. 6 is a view illustrating an example table using a likelihood.

FIG. 7 is a hardware block diagram illustrating an example computer for implementing functions of various information processing apparatuses, such as a client and a server.

### MODE FOR CARRYING OUT THE INVENTION

Hereinafter, embodiments according to the present disclosure will be described in detail on the basis of the drawings. Note that an information processing apparatus and an information processing method of the present invention are not limited to the embodiments. Furthermore, redundant description in each of the following embodiments is omitted by assigning the same signs to the same parts.

The present disclosure will be described in the following item order.

1. Description of AI

2. Embodiment
2-1. Description of background
2-2. Description of insurance system
2-3. Flow of insurance system

3. Modification 1 of insurance system

4. Modification 2 of insurance system

5. Other embodiments

6. Effects according to the present disclosure

7. Hardware configuration

### 1. Description of AI

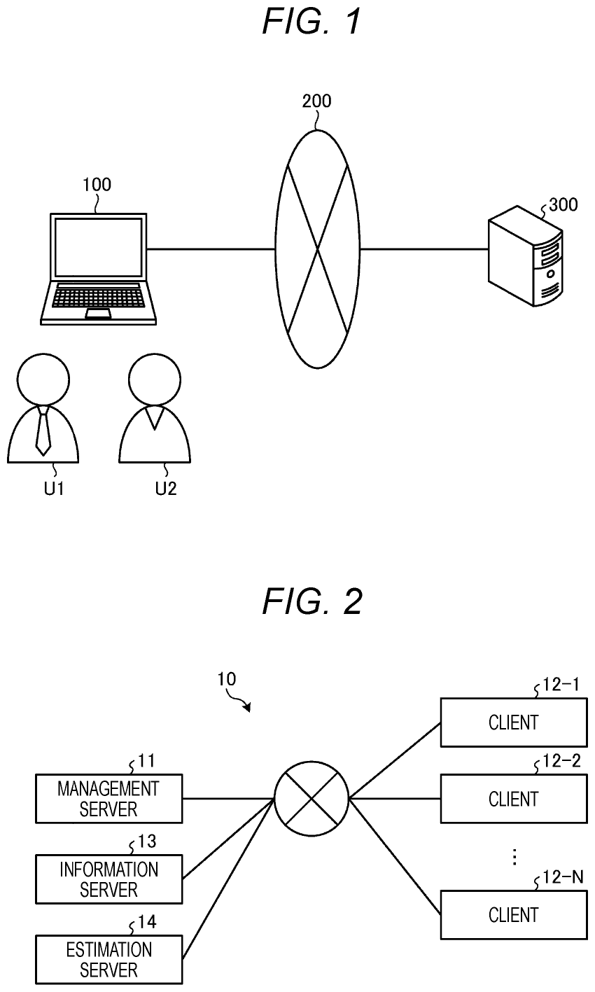

In description of embodiments, AI means a system using machine learning. FIG. 1 is a view illustrating an outline of an example pathological AI system, which is a kind of AI. For example, FIG. 1 is a schematic view of a pathological AI system.

The pathological AI system is a system including a client 100 and a server 300. The client 100 is an information processing apparatus capable of communicating with the server 300 through a network 200, and is, for example, a personal computer (PC) or a smartphone. The server 300 is an information processing apparatus capable of executing a pathological AI program that estimates a condition of a patient U2 on the basis of an input pathology image of the patient U2, and outputs an estimated result, and is, for example, a server PC. A PC is an information processing apparatus mounted with circuits including, for examples, a central processing unit (CPU), a graphics processing unit (GPU), a random access memory (RAM), and a read only memory (ROM).

A doctor U1 inputs a pathology image of a target patient U2 to the client 100, and inputs the pathology image to the server 300 through the network 200. At this time, the doctor U1 may input the pathology image to the client 100, or the doctor U1 may operate the client 100 to cause the server 300 to acquire the pathology image from an external device.

The server 300 estimates a condition of the patient U2 (for example, whether or not the patient U2 has a cancer) on the basis of the input pathology image of the patient U2, and outputs an estimated result to the client 100. The doctor U1 uses the estimated result as reference information and finally refers to the knowledge of the doctor U1, and tells his/her diagnostic result to the patient U2.

The pathology AI program is a program that estimates the condition of the patient U2 on the basis of the input pathology image of the patient U2, and is, for example, a program using machine learning. The pathology AI program is generated through, for example, the following processes. The server 300 generates parameters through training with input of learning data including a plurality of pathology data pieces, in each of which a pathology image and a condition of a patient (diagnostic result) are linked with each other, to a predetermined machine learning algorithm. The server 300 sets parameters of an inference algorithm on the basis of the generated parameters. The inference algorithm outputs an estimated result of the condition of the target patient and its likelihood upon input of pathology data of the target patient (input data). The likelihood is a probability representing how much it is likely that the estimated result is correct. The pathology AI program is a program equipped with the inference algorithm. Note that parameter generation performed through machine learning may be performed by an information processing apparatus other than the server 300.

The machine learning algorithm and the inference algorithm are machine learning algorithms using, for example, a multilayer neural network. The multilayer neural network is an algorithm including at least an input layer, a plurality of intermediate layers and an output layer. Each layer includes a plurality of items to which parameters are set. By inputting learning data (at least data pieces to which classification labels are assigned), the multilayer neural network automatically adjusts parameters of each layer to fit the classification labels of the learning data. Examples of the machine learning algorithm and the inference algorithm include a convolution neural network (CNN), a recurrent neural network (RNN) and a capsule-neural network (Capsule-NN).

Although the pathological AI system is described above, an AI algorithm is not limited to that in a pathological field. The AI algorithm is only required to be an algorithm that estimates a condition of a patient on the basis of patient's information that is input data, using machine learning. For example, parameters are generated through training with input of learning data including a plurality of time-series patient data pieces, in each of which heartbeat data of a patient for a predetermined time length and a diagnostic result of the patient (for example, are linked with each other, to a predetermined machine learning algorithm. Parameters of an inference algorithm are set on the basis of the generated parameters. Through the processes, a heartbeat AI algorithm that estimates a condition of a patient on the basis of heartbeat data of the patient can be generated. In this case, the machine learning algorithm and the inference algorithm are algorithms using a multilayer neural network, and each are preferably, for example, a long short-term memory (LSTM) or an RNN. Alternatively, the AI algorithm may be ultrasonic diagnosis AI that determines whether or not a cancer (malignant tumor) is present in an ultrasonic image. For example, parameters are generated through training with input of learning data including a plurality of data pieces, in each of which an ultrasonic image and a label indicating whether or not a cancer is present in the ultrasonic image are linked with each other, to a predetermined machine learning algorithm. Parameters of an inference algorithm are set on the basis of the generated parameters. Through the processes, an ultrasonic diagnosis AI algorithm that estimates, on the basis of an ultrasonic image of a patient, whether or not a cancer is present in an area measured by an ultrasonic wave can be generated. In this case, the machine learning algorithm and the inference algorithm are algorithms using a multilayer neural network, and each are preferably, for example, a CNN or a Capsule-NN.

### 2. Embodiment 1

An example case where doctors use AI will be described according to an aspect of an embodiment.

[2-1. Description of Background]

When a doctor diagnoses a disease (illness) of a patient in a clinical field, the doctor makes the diagnosis in consideration of a variety of information about the patient. A doctor estimates a disease of a patient, for example, by taking the patient's temperature with an inspection device, and further observing swelling and color of the patient's throat. Then, the doctor finally tells an estimated result of the doctor to the patient as a diagnostic result. Use of AI as a second opinion when telling the diagnostic result has been expected to improve accuracy of diagnosis. For example, a pathologist may overlook an abnormal part of cells due to necessity of observing a large amount of pathology images in a pathological field. With use of AI, an effect of decreasing such oversights has been expected. Furthermore, decreasing doctors' burdens has been also expected for the future, by proposing, to doctors, only the information needs to be confirmed by doctors through screening of patients' information by using AI.

However, although accuracy of diagnosis by AI has been increasing year by year, AI does not have 100% accuracy. Thus, AI may make wrong diagnosis. For example, pathological estimation with pathological AI may estimate a normal cell to be an abnormal cell, or an abnormal cell to be a normal cell.

Under the operation in which a doctor confirms an estimated result of AI and makes final diagnosis, the diagnosis may be wrong, for example, when a doctor overlooks incorrect estimation of AI. That is, a doctor makes wrong diagnosis on the condition of a patient. Since a doctor makes final decision at this time, it is prescribed in many countries that the doctor is responsible for the wrong diagnosis.

However, accuracy of estimation (accuracy of diagnosis) by AI is often higher than that by doctors with a low skill (inexperienced doctors). That is, for cases to which AI gives wrong estimation, it is assumed that even human doctors may often make wrong diagnosis Furthermore, overturning the estimated result by AI puts the doctors with a low skill at a risk of future lawsuits. Due to overturning a first opinion by AI that has higher accuracy of diagnosis than a doctor himself/herself, in a case where a second opinion by the doctor is wrong (in other words, in a case where the estimated result by AI is correct), why the doctor did not trust AI is considered problematic on a lawsuit. Consequently, the doctors with a low skill may tend to accept the estimated result by AI as it is. Thus, the estimated result by AI is likely to be told to a patient as a diagnostic result with insufficient confirmation by doctors.

Furthermore, accuracy of estimation by AI is often lower than that by doctors with a high skill (experienced doctors). Unfortunately, in a case where AI is used for screening, that is, AI is used as a system for extracting parts to be checked by doctors, it is assumed that the doctors cannot make confirmation in the first place. In a case where doctors are responsible for the estimated result by AI even in this case, doctors face a risk of lawsuits for the wrong diagnosis.

Thus, in a case where it is prescribed that doctors are responsible for the estimated result by AI, doctors can enjoy merits, such as decreasing oversights of diseases or the number of objects to be diagnosed, but doctors may face a risk of lawsuits. Consequently, it has been difficult to prompt utilization of AI.

[2-2. Description of Insurance System]

An insurance system that can promote utilization of AI is described with reference to FIG. 2. FIG. 2 is a view illustrating an outline of an example insurance system.

An insurance system 10 includes a management server 11 and clients 12-1, 12-2, . . . , and 12-N. Hereinafter, when the clients 12-1, 12-2, . . . , and 12-N are described without distinction, they may be referred to as the “clients 12”. Furthermore, an information server 13 and an estimation server 14 are connected to a network. Each of the clients 12 is an information processing apparatus capable of communicating with the management server 11 through the network, and is, for example, a personal computer (PC) or a smartphone. For example, the client 12-N may be a smartphone capable of communicating with the management server 11 through the network. The management server 11 is an information processing apparatus capable of executing an insurance coverage determination program having a function of calculating insurance premium on the basis of input information acquired from clients, or information about the information server 13 and the estimation server 14, and is, for example, a server PC. The information server 13 is an information processing apparatus that integrates and stores information about a hospital, such as medical records, surgery records, or diagnosis records of patients, and is, for example, a server PC. An inference server is an information processing apparatus capable of executing an AI program that estimates a condition of a patient on the basis of input patient's information, and outputs an estimated result, and is, for example, a server PC.

In the insurance system 10, the management server 11 executes the insurance coverage determination program, and the insurance coverage determination program acquires a type of a condition of a patient (first patient condition) estimated by a machine learning algorithm on the basis of patient's data (first patient data) from at least one of the clients 12, the information server 13, or the estimation server 14. Furthermore, the insurance coverage determination program acquires a type of a condition of the patient (second patient condition) estimated by a method different from the machine learning algorithm on the basis of data including the first patient data, from at least one of the clients 12, the information server 13, or the estimation server 14. Moreover, the insurance coverage determination program acquires a third patient condition estimated on the basis of second patient data measured after measurement time of the first patient data, from at least one of the clients 12, the information server 13, or the estimation server 14.

The insurance coverage determination program determines whether or not insurance coverage is available on the basis of the first patient condition, the second patient condition, and the third patient condition. The determination of whether or not insurance coverage is available is made as follows. It is determined that insurance coverage is available in a case where a relationship between the first patient condition, the second patient condition, and the third patient condition satisfies a predetermined condition, while it is determined that insurance coverage is unavailable in a case where the relationship fails to satisfy the predetermined condition.

The predetermined condition may be data in which the relationship between the first patient condition, the second patient condition, and the third patient condition and whether or not insurance coverage is available are linked, and is, for example, a condition stored in advance as a table in the management server 11.

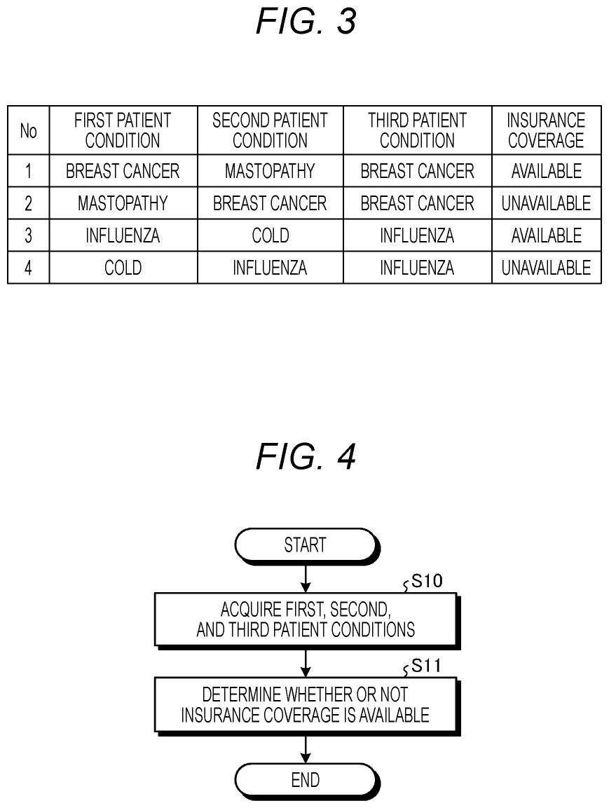

FIG. 3 is a view illustrating an example table. The insurance coverage determination program searches the table for a row that each of the first patient condition, the second patient condition, and the third patient condition that has been acquired matches, reads data from the column of insurance coverage indicated in the same row, and determines whether or not insurance coverage is available.

In the table, for example, in row 1 indicated in the column of No, insurance coverage is set to be available in a case where the first patient condition differs from the second patient condition, and the third patient condition matches the first patient condition. This means that a condition of a patient estimated by AI differs from a condition of the patient estimated by a doctor, and the third patient condition, which is a result diagnosed when a symptom becomes easier to be estimated as time has passed, matches the first patient condition. This is a situation where estimation by AI is correct while estimation by the doctor is wrong, that is, the doctor makes wrong diagnosis. Under the situation, the insurance coverage determination program is executed on the basis of an application by the doctor, and determines whether or not the situation is covered by insurance. Accordingly, financial risk (risk of a lawsuit from a patient) arising from not believing AI blindly is reduced. That is, it becomes easier for doctors to make diagnosis by their own thought considering a possibility of wrong diagnosis by AI, and to utilize AI since they do not need to blindly believe AI that may make wrong diagnosis.

At this time, it may be determined whether each disease of the first patient condition, second patient condition, and the third patient condition is benign or malignant, in the table. For example, the insurance coverage may be set to be unavailable in a case where the first patient condition and the third patient condition are benign and the second patient condition is malignant, while the insurance coverage may be set to be available in a case where the first patient condition and the third patient condition are malignant and the second patient condition is benign. That is, determining whether a disease is benign or malignant allows fuzzy determination even if the disease estimated as the first patient condition differs from the disease estimated as the third patient condition. Because of a time difference between the time when the first patient data is measured and the time when the second patient data is measured, the disease may progress or provoke another disease. Allowing fuzzy determination on the basis of disadvantages that a patient suffers makes insurance coverage more flexible.

The insurance system, in a case where a patient is disadvantaged because, for example, AI makes wrong diagnosis or wrong screening and a doctor also fails to find such errors, can reduce a financial burden of the doctor or an organization to which the doctor belongs by covering by insurance. Accordingly, the insurance system decreases demerits of use of AI by doctors so that it can be expected to achieve active use of AI.

[2-3. Flow of Insurance System]

An example flow of the insurance system is described below with reference to FIG. 4. FIG. 4 is an example flowchart of the insurance system.

In step S10, the insurance coverage determination program executed on the management server 11 acquires a type of a condition of a patient (first patient condition) estimated by the machine learning algorithm on the basis of first patient data from at least one of the clients 12, the information server 13, or the estimation server 14. Furthermore, the insurance coverage determination program acquires a type of a condition of the patient (second patient condition) estimated by a method different from the machine learning algorithm used for estimation of the first patient condition on the basis of data including the first patient data from at least one of the clients 12, the information server 13, or the estimation server 14. Furthermore, the insurance coverage determination program acquires a type of a condition of the patient (third patient condition) estimated on the basis of second patient data measured at the time after the time when the first patient data is measured, from at least one of the clients 12, the information server 13, or the estimation server 14.

In step S11, the insurance coverage determination program executed on the management server 11 determines whether or not insurance coverage is available on the basis of a relationship between the first patient condition, the second patient condition, and the third patient condition. The relationship may be determined by using a table stored in advance, or by a predetermined formula stored in advance.

### 3. Modification 1 of Insurance System

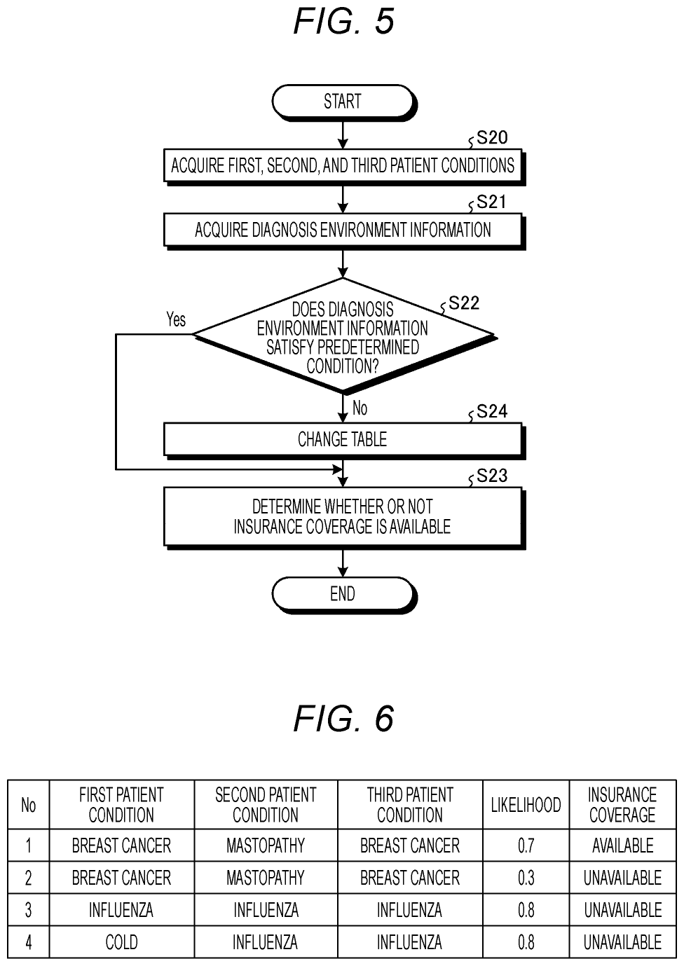

A modification of the above-described insurance system is described below with reference to FIG. 5. In the present modification, an example using diagnosis environment information for determining whether or not insurance coverage is available is further described. Note that a configuration of an insurance system is similar to the above. FIG. 5 is an example flowchart of the modification.

Step S20 is similar to step S10. In step S21, the management server 11 acquires diagnosis environment information. The diagnosis environment information is information indicating an environment where a doctor estimates a second patient condition, and is, for example, a fatigue level of a doctor (the number of times the doctor blinks per a predetermined time unit, or the like). Furthermore, the diagnosis environment information may be the number of cases a doctor handles per a day, a time taken for diagnosis, line-of-sight information regarding whether or not a doctor sees a part of first patient data significant for diagnosis, or the like. In a case where a doctor makes estimation after the doctor confirms a result by AI, time period information, line-of-sight information, or mouse movement information from the time when the result by AI is displayed till the time when the doctor makes the estimation, may be used as the diagnosis environment information, as well. The diagnosis environment information may also include brightness of lighting, a type of a camera, or the like. Furthermore, the diagnosis environment information may be a behavioral pattern of a doctor, a type of a portable terminal of a doctor, or the like.

In step S22, the management server 11 determines whether or not the diagnosis environment information satisfies a predetermined condition. For example, the management server 11 determines whether or not a fatigue level of a doctor is smaller than a predetermined threshold value. In a case where the predetermined condition is satisfied, the processing proceeds to step S23, and in a case where the predetermined condition is not satisfied, the processing proceeds to step S24.

In step S23, the management server 11 makes a similar determination to step S11. In step S24, the management server 11 changes the condition of insurance coverage used in step S11 (for example, a table), to a different condition of insurance coverage (for example, another table). After the change of the condition of insurance coverage, the processing proceeds to step S23.

By thus using the diagnosis environment information, in a case where a doctor is in an environment that obviously causes wrong diagnosis, an insurance coverage rule can be changed. For example, in a case where a doctor presents a result by AI as it is as his/her own estimated result shortly after the result by AI is displayed, an amount of money the doctor should pay in a lawsuit may be more due to responsibility of the doctor, which is considered obviously serious. Then, a flexible change of insurance coverage by using the diagnosis environment information makes calculation for availability of insurance coverage and calculation of insurance money more suitable.

### 4. Modification 2 of Insurance System

A modification 2 of the insurance system is described with reference to FIG. 6. In the present modification, an example further using likelihood information for determining whether or not insurance coverage is available is described. Note that a configuration of an insurance system is similar to the above. FIG. 6 is a view illustrating an example table using a likelihood.

When classification using machine learning is performed, classification and information about its likelihood are output as an output result. That is, a result of the classification, and probability information about how much it is likely that the classification is correct are output. The information about a likelihood may be acquired by the management server 11, and used for determining whether or not insurance coverage is available.

The management server 11 makes determination of step S11 using a table including the information about a likelihood as indicated in FIG. 6. Accordingly, in a case where a likelihood output by AI is smaller than a predetermined threshold value, in other words, a case where reliability of a patient condition estimated by AI is low, the first patient condition is less valuable information to a doctor. Thus, in the case where a likelihood is smaller than a predetermined threshold value, a doctor has little need to see the information about the first patient condition. Furthermore, for the low reliability of information about the first patient condition, the first patient condition is not suitable as a basis for determining whether or not insurance coverage is available. Consequently, in a case where a likelihood is low, insurance coverage may be determined to be unavailable.

Although the insurance system for medical AI used by doctors has been described in the above embodiment, the insurance system may also be applicable to medical AI used by patients. For medical AI used by patients, the second patient condition is a condition of a patient selected by the patient with reference to the first patient condition that is estimated by AI. Furthermore, the third patient condition is a patient condition diagnosed by a doctor.

A case is also assumed where it is judged that AI developers and manufacturers are responsible instead of doctors. In such a case, covered objects by insurance in the insurance system according to the present embodiment may be AI developers and manufacturers. That is, insurance money may be paid to the AI developers and manufacturers.

### 5. Other Embodiments

Processes according to the embodiment or the modifications described above may be implemented in various different forms (modifications) other than the above embodiment or modifications.

Furthermore, out of the processes described in each embodiment above, all or a part of the processes that is described as being performed automatically may be performed manually, and also, all or a part of the processes that is described as being performed manually may be performed automatically by known methods. Note that information including processing procedures, specific names, and various types of data and parameters described in the above description and indicated in drawings may be changed optionally unless otherwise specified. For example, various types of information indicated in each figure are not limited to the information illustrated in the figure.

Note that each component in each apparatus illustrated in the figures is functionally conceptual, and not necessary to be physically configured as illustrated in the figures. That is, a specific form of distribution/integration of the apparatuses is not limited to the one illustrated in the figures, and all or a part thereof may be distributed/integrated and configured functionally or physically in any unit in accordance with various loads and usage conditions.

Furthermore, each embodiment or modification described above may be optionally combined as long as no processing content is contradictory.

Note that the effects described in the description are just an example and effects are not limited thereto, and other effects may be exerted.

### 6. Effects According to the Present Disclosure

As described above, the medical insurance method according to the present disclosure includes a step of acquiring, as a first patient condition, a type of a condition of a patient estimated through input of first patient data of the patient to a predetermined machine learning model, a step of acquiring, as a second patient condition, a type of a condition of the patient estimated by a method different from the predetermined machine learning model on the basis of data including the first patient data, a step of acquiring a third patient condition estimated on the basis of second patient data measured after measurement time of the first patient data, and a step of determining whether or not insurance coverage is available on the basis of the first patient condition, the second patient condition, and the third patient condition.

With these steps, the medical insurance method according to the present disclosure can cover disadvantages of users or developers arising from wrong diagnosis by AI (achieve dispersion of financial disadvantages) by insurance, which can promote utilization of AI in a medical field. For example, in a case where doctors are responsible for wrong diagnosis by AI, doctors cannot use AI unless they can trust AI. In a case where, for example, AI diagnoses cancer even if a doctor thinks that it is not cancer, and the doctor adopts the diagnosis by AI, the patient's burden (inspection burden or financial burden) increases due to wrong diagnosis by AI. Furthermore, in a case where patients are responsible for wrong diagnosis by AI, and AI makes wrong diagnosis indicating that a patient does not have the flu (the patient is positive for the flu), the patient does not go to hospital, which may worsen the flu. However, the medical insurance method according to the present disclosure can cover disadvantages of users or developers arising from wrong diagnosis by AI by insurance as described above. Thus, the medical insurance method according to the present disclosure can achieve dispersion of disadvantages arising from wrong diagnosis by AI.

Furthermore, in the medical insurance method, whether or not insurance coverage is available is determined on the basis of a relationship between the first patient condition, the second patient condition, and the third patient condition. With this step, whether or not the insurance coverage is available can be accurately determined by the medical insurance method.

Furthermore, the relationship is determined by using information about a lookup table stored in advance. With this step, whether or not the insurance coverage is available can be accurately determined by the medical insurance method.

Furthermore, in the medical insurance method, whether or not insurance coverage is available is determined on the basis of whether or not the first patient condition matches the third patient condition in a case where the first patient condition differs from the second patient condition. With this step, whether or not the insurance coverage is available can be accurately determined by the medical insurance method.

Furthermore, in the medical insurance method, whether each of the first patient condition, the second patient condition, and the third patient condition is a benign disease or a malignant disease is determined by using table information, and the insurance coverage is determined available in a case where the first patient condition differs from the second patient condition, and the third patient condition and the first patient condition are malignant. With this step, whether or not the insurance coverage is available can be accurately determined by the medical insurance method.

Furthermore, in the medical insurance method, whether or not insurance coverage is available is determined by using diagnosis environment information. With this step, whether or not the insurance coverage is available can be accurately determined by the medical insurance method.

Furthermore, the diagnosis environment information includes information about environment where a doctor diagnoses or patient data is acquired. With this step, whether or not the insurance coverage is available can be accurately determined by the medical insurance method.

Furthermore, the diagnosis environment information includes at least one of a time period taken for decision by a doctor himself/herself, a time period taken from the time when a result by AI is output till the time when confirmation is pressed, line-of-sight information, mouse movement information, a fatigue level of a doctor, brightness of lighting, or a type of a camera. With this step, whether or not the insurance coverage is available can be accurately determined by the medical insurance method.

Furthermore, the diagnosis environment information includes at least one of a behavioral pattern, the number of times a doctor blinks, or a type of a portable terminal of a doctor. With this step, whether or not the insurance coverage is available can be accurately determined by the medical insurance method.

Furthermore, in the medical insurance method, whether or not the insurance coverage is available is determined by using information about a likelihood. With this step, whether or not the insurance coverage is available can be accurately determined by the medical insurance method.

### 7. Hardware Configuration

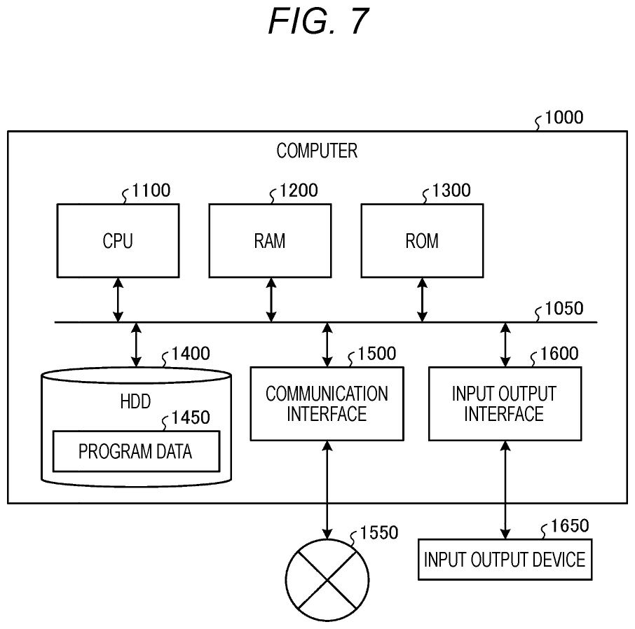

Various information processing apparatuses including the clients 100, and 12, the server 300, the management server 11, the information server 13, and the estimation server 14 according to each embodiment or modification described above are implemented by, for example, a computer 1000 that has a configuration illustrated in FIG. 7. FIG. 7 is a hardware block diagram illustrating an example of the computer 1000 for implementing functions of various information processing apparatuses, such as a client and a server. Hereinafter, the server 300 according to the embodiment is described as an example. The computer 1000 includes a CPU 1100, a RAM 1200, a read only memory (ROM) 1300, a hard disk drive (HDD) 1400, a communication interface 1500, and an input output interface 1600. The parts of the computer 1000 are connected through a bus 1050.

The CPU 1100 operates on the basis of programs stored in the ROM 1300 or the HDD 1400, and controls each part. For example, the CPU 1100 deploys programs stored in the ROM 1300 or the HDD 1400 to the RAM 1200, and performs processes that correspond to various programs.

The ROM 1300 stores a boot program such as a basic input output system (BIOS) executed by the CPU 1100 upon starting the computer 1000, programs depending on the hardware of the computer 1000, and the like.

The HDD 1400 is a computer-readable recording medium that records programs executed by the CPU 1100, data used by the programs, and the like in a non-transient manner. The HDD 1400 is, specifically a recoding medium for recording an information processing program according to the present disclosure that is an example of a program data 1450.

The communication interface 1500 is an interface for connecting the computer 1000 to an external network 1550 (for example, the Internet). For example, the CPU 1100 receives data from other devices and transmits data that the CPU 1100 generates to other devices through the communication interface 1500.

The input output interface 1600 is an interface for connecting an input output device 1650 and the computer 1000. For example, the CPU 1100 receives data from input devices, such as a keyboard or a mouse through the input output interface 1600. Furthermore, the CPU 1100 transmits data to output devices, such as a display, a speaker, or a printer through the input output interface 1600. Furthermore, the input output interface 1600 may function as a media interface for reading programs and the like recorded in a predetermined recording medium (medium). The medium includes, for example, an optical recording medium, such as a digital versatile disc (DVD) and a phase change rewritable disk (PD), a magneto-optical recording medium, such as a magneto-optical disk (MO), a tape medium, a magnetic recording medium, a semiconductor memory, or the like.

For example, in a case where the computer 1000 functions as the server 300 according to the embodiment, the CPU 1100 of the computer 1000 implements functions of the control unit and the like of the server 300 by executing an information processing program loaded in the RAM 1200. Furthermore, the HDD 1400 stores an information processing program according to the present disclosure and data in a memory unit of the server 300. Note that although the CPU 1100 reads the program data 1450 from the HDD 1400 and executes the programs, the CPU 1100 may alternatively acquire these programs from another device through the external network 1550.

The present technique may also have the following configurations.

(1)

A medical insurance method including

a step of acquiring, as a first patient condition, a type of a condition of a patient estimated through input of first patient data of the patient to a predetermined machine learning model,

a step of acquiring, as a second patient condition, a type of a condition of the patient estimated by a method different from the predetermined machine learning model on the basis of data including the first patient data,

a step of acquiring a third patient condition estimated on the basis of second patient data measured after measurement time of the first patient data, and

a step of determining whether or not insurance coverage is available on the basis of the first patient condition, the second patient condition, and the third patient condition.

(2)

The medical insurance method according to (1),

in which whether or not insurance coverage is available is determined on the basis of a relationship between the first patient condition, the second patient condition, and the third patient condition.

(3)

The medical insurance method according to (2),

in which the relationship is determined by using information about a lookup table stored in advance.

(4)

The medical insurance method according to any one of (1) to (3),

in which whether or not insurance coverage is available is determined on the basis of whether or not the first patient condition matches the third patient condition in a case where the first patient condition differs from the second patient condition.

(5)

The medical insurance method according to any one of (1) to (4),

in which whether each of the first patient condition, the second patient condition, and the third patient condition is a benign disease or a malignant disease is determined by using table information, and the insurance coverage is determined available in a case where the first patient condition differs from the second patient condition, and the third patient condition and the first patient condition are malignant.

(6)

The medical insurance method according to any one of (1) to (5),

in which whether or not insurance coverage is available is determined by using diagnosis environment information.

(7)

The medical insurance method according to (6),

in which the diagnosis environment information includes information about environment where a doctor diagnoses or patient data is acquired.

(8)

The medical insurance method according to (6) or (7),

in which the diagnosis environment information includes at least one of a time period taken for decision by a doctor himself/herself, a time period taken from the time when a result by AI is output till the time when confirmation is pressed, line-of-sight information, mouse movement information, a fatigue level of a doctor, brightness of lighting, or a type of a camera.

(9)

The medical insurance method according to any one of (6) to (8),

in which the diagnosis environment information includes at least one of a behavioral pattern, the number of times a doctor blinks, or a type of a portable terminal of a doctor.

(10)

The medical insurance method according to any one of (1) to (9),

in which whether or not insurance coverage is available is determined by using information about a likelihood.

### REFERENCE SIGNS LIST

10 Insurance system
11 Management server
12-1, 12-2, . . . , 12-N Client
13 Information server
14 Estimation server
100 Client
200 Network
300 Server

1. A medical insurance method comprising:
a step of acquiring, as a first patient condition, a type of a condition of a patient estimated through input of first patient data of the patient to a predetermined machine learning model;
a step of acquiring, as a second patient condition, a type of a condition of the patient estimated by a method different from the predetermined machine learning model on a basis of data including the first patient data;
a step of acquiring a third patient condition estimated on a basis of second patient data measured after measurement time of the first patient data; and
a step of determining whether or not insurance coverage is available on a basis of the first patient condition, the second patient condition, and the third patient condition.

2. The medical insurance method according to claim 1,
wherein whether or not insurance coverage is available is determined on a basis of a relationship between the first patient condition, the second patient condition, and the third patient condition.

3. The medical insurance method according to claim 2,
wherein the relationship is determined by using information about a lookup table stored in advance.

4. The medical insurance method according to claim 1,
wherein whether or not insurance coverage is available is determined on a basis of whether or not the first patient condition matches the third patient condition in a case where the first patient condition differs from the second patient condition.

5. The medical insurance method according to claim 1,
wherein whether each of the first patient condition, the second patient condition, and the third patient condition is a benign disease or a malignant disease is determined by using table information, and insurance coverage is determined available in a case where the first patient condition differs from the second patient condition, and the third patient condition and the first patient condition are malignant.

6. The medical insurance method according to claim 1,
wherein whether or not insurance coverage is available is determined by using diagnosis environment information.

7. The medical insurance method according to claim 6,
wherein the diagnosis environment information includes information about environment where a doctor diagnoses or patient data is acquired.

8. The medical insurance method according to claim 6,
wherein the diagnosis environment information includes at least one of a time period taken for decision by a doctor himself/herself, a time period taken from time when a result by AI is output till time when confirmation is pressed, line-of-sight information, mouse movement information, a fatigue level of a doctor, brightness of lighting, or a type of a camera.

9. The medical insurance method according to claim 6,
wherein the diagnosis environment information includes at least one of a behavioral pattern, the number of times a doctor blinks, or a type of a portable terminal of a doctor.

10. The medical insurance method according to claim 1,
wherein whether or not insurance coverage is available is determined by using information about a likelihood.
